(19) United States
(12) Patent Application Publication
(10) Pub. No.: US 2024/0398515 A1
(43) Pub. Date: Dec. 5, 2024

(54) TEMPERATURE CONTROL DRILLING METHOD USING LARGE SURFACE DRILLS MADE OF HIGH THERMAL CONDUCTIVITY MATERIALS

(71) Applicant: Zvi FUDIM, Dollard-Des-Ormeaux (CA)

(72) Inventor: Zvi FUDIM, Dollard-Des-Ormeaux (CA)

(21) Appl. No.: 18/694,871

(22) PCT Filed: Sep. 22, 2022
(86) PCT No.: PCT/CA2022/051407

§ 371 Date: Mar. 22, 2024

Related U.S. Application Data
(60) Provisional application No. 63/261,473, filed on Sep. 22, 2021.

(51) Int. Cl. A61C 8/00; A61C 1/08

(52) U.S. Cl. CPC A61C 8/0089; A61C 1/084

(57) ABSTRACT

The present invention is about a method and kits that use surgical drills with high capacity to absorb heat due to their large surface area and their thermodynamic material properties. The present invention also discloses a “Crown Down” drilling sequence which is comprised of drilling the bone using not more than two drills, saving time and the number of drills needed to perform an osteotomy.

### FIELD OF THE INVENTION

This invention relates in general to a temperature control drilling method for drilling a borehole in the jaw bone, maxillary or mandibular, and in particular to create an osteotomy for root-form intraosseous dental implants. This method is referred to as the “Crown Down” drilling method.

This invention also relates in general to rotary drill bits used in drilling a borehole in the jaw bone, maxillary or mandibular, and in particular to create an osteotomy for root-form intraosseous dental implants.

### BACKGROUND OF THE INVENTION

It is known that the dental implantation of root form dental implants is a treatment of choice in replacing missing teeth. To replace a missing tooth there is a need to drill a cylindrical or tapered hole followed by insertion of an artificial root made of titanium or zirconium. In recent years dental implant treatments have had relatively high success rates; however, in some cases there are complications and even failures. One of the biggest challenges in dental implantation is preventing the jaw bone from over heating during the drilling process and providing the implant with a sufficient initial stability before it can completely integrate in the bone. Many different drill geometries are presently in use; some of them are twisted or straight, with two, three, or more flutes. Most of the drills used for dental implants are made of HSS (HIGH SPEED STEEL) but other materials such as zirconium are in use too.

Since the bone tissue can sometimes be very hard, the drills are generally driven by special high torque micro motors. If used with a 1:20 reduction handpiece, they can deliver up to 1000 N/cm. The high torque is needed to overcome the resistance of hard bone, especially in cases of important alveolar bone resorption. The alveolar bone is softer than the rest of the jaw bone. The greater is the alveolar bone resorption, the harder is the bone. Drilling hard bone with high torque, on high RPM, and with a lot of force, results in a great amount of friction and heat. The rise of the temperature of the bone to 47° C. causes bone necrosis and altering of the implant integration. One of the solutions to prevent the bone from being overheated is a liquid coolant; however, in many cases this is not sufficient. The jaw bone's hardness was classified into 4 categories by Dr. Karl Mish: D4, the softest class of bone, up to D1, the hardest class of bone. D1 class bone is mostly found in the lower jaw while D4 class bone is mainly found in the upper jaw in the maxillary tuberosity areas. Due to the varieties of the bone density levels, dental implantation requires different approaches for each type of bone.

Most of the dental implants have self-tapping features due to their threaded surface. Implant drills are designed according to these implant capacities to tap a thread in the bone and even to condense the bone matter. Too hard bone or too small diameter of the drilling may alter the self-tapping function preventing the implant to be fully inserted.

Bone tissue is an extremely abrasive material which causes fast wear of HSS standard drills. HSS drills become dull after approximately 20 cycles, after which it is recommended that they be replaced by most implant companies.

It is known in dentistry to drill jaw bone for root shape dental implants using rotary instruments such as twisted drill bits. The drill bits for dental implantation are organized in a form of surgical kits. The drills are organized in ascending order from smaller to larger diameter (such as from 1.8 mm to a maximum of 6 mm in increments of approximately 0.5 mm). The drills are equipped with transverse depth markings, usually at 6, 8, 10, 11.5, 13 and 16 mm, depending on the implant length of each system. Drilling in ascending order with small increments often makes it hard to control the depth due to lack of resistance and excessive feed rate. The drill shape can vary from simple or multiple cylinder, taper, or a combination of both.

Conventional prior art techniques consist of drilling first with the smallest diameter (e.g. 2 mm) and then increasing the diameter of the drills incrementally until reaching the final diameter (e.g. 6 mm). They also consist of drilling to the full depth during the entire osteotomy. The diameter of the last drill is smaller than the diameter of the implant in order to create a tight fit between the cervical part of the implant and the cortical bone area. Most of the implants do not have threads on their cervical part, which sometimes makes the implant insertion difficult, especially when the bone in the cortex area is very hard and the medullar area is very soft. In these cases, the coronal resistance forces exceed the threading force so as to move the implant in an apical direction, resulting in incomplete implant insertion. Consequently, the self-taped threads break and the implant fails to achieve the minimum required stability needed to integrate.

As mentioned, conventional prior art techniques use drills made of HSS (High Speed Steel) with a speed range between 800 to 1200 RPM, and liquid cooling. Conventional implant drilling kits are comprised of multiple drill bits for ascending incremental drilling.

A study published in “Clinical Implant Dentistry and Related Research” in 2013 finds that drilling with a single drill doesn't generate more heat than drilling with multiple drills.

Another study published in 2018 in the Journal of Dental Science examines an experiment where a simplified drilling technique was used (the first drill being the smallest and the second final drill being the biggest). This study does not find any impact on implant stability compared to drilling with the entire series of drills using standard incremental method.

FIG. 1 presents a prior art drilling chart comprised of one pilot drill 11 with a 2.3 mm diameter and a set of five other drills 12 with an incremental increase in diameter between each of them; 2.5 mm, 2.8 mm, 3.1 mm, 3.6 mm, and 4.2 mm. The chart of FIG. 1 also shows two stages of the implantation motorized and manual 13. The implant 14 diameter (5.2 mm) is larger than the last drill by 1.0 mm.

FIG. 2 explains the prior art “incremental” technique wherein each diameter is used to drill the entire depth starting from the cortical bone 23 and continuing in the medullar bone 24. The depth of the drilling 22 for each of drills I, II, III, and IV is always the same, while the diameter D1, D2, D3, and D4, is increasing.

### SUMMARY OF THE INVENTION

In accordance with the present invention, there is provided:
1. A method of drilling bone for a dental implant (58) having a threaded portion and non-threaded portion, the method comprising the steps of:
   drilling a cortical area (53) with a first drill (51) up to a first depth, said first drill (51) having a diameter (D1) approximately equal to a widest diameter of the dental implant (58) and said first depth corresponding to a length of the non-threaded portion of the dental implant (58);
   drilling a borehole (54) into said cortical area (53) with a second drill (52) up to a second depth, said second drill (52) having a smaller diameter (D2) than the first drill (51) that is approximately equal to a difference between the widest diameter and a depth of threads of the dental implant (58), and said second depth corresponding to a length of the threaded portion (50) of the dental implant (58), such that the sum of the first depth and the second depth is approximately equal to the total length of the dental implant (58).
2. The method of item 1 or 2, wherein, once the dental implant (58) is inserted, the cortical bone and the cervical end (59) of the implant experience less stress caused by pressing against each other.
3. The method of any one of items 1 to 2, wherein the first drill (51) and/or the second drill (52) have larger surface area to better absorb the heat resulting from the friction between the bone, the bone chips, and the active end of the drill.
4. The method of any one of items 1 to 3, wherein the first drill (51) and/or the second drill (52) are made of tungsten carbide.
5. The method of any one of items 1 to 4, wherein the first drill (51) and/or the second drill (52) operate with a speed of about 200 RPM.
6. The method of any one of items 1 to 5, wherein the method is free of irrigation.
7. The method of any one of items 1 to 6, wherein the first drill (51) comprises a first stopper (70) and/or the second drill (52) comprises a second stopper (75).
8. The method of item 7, wherein a guiding tube (80) is used in combination with the stoppers (70, 75), to guide the drills (51, 52) during drilling.
9. A kit comprising the first drill (51) and the second drill (52) as defined in any one of items 1 to 8.
10. A kit comprising the first drill (51) and the second drill (52) as defined in item 7, further comprising a guiding tube (80), wherein each stopper (70, 75) is configured to get slidably pushed along the guiding tube (80) during use of the drills (51, 52), and wherein an external surface of each stopper (70, 75) is configured to guide the drills (51, 52) along an inner surface of the guiding tube (80) during use thereof.
11. A method of drilling bone for a non-threaded dental implant, the method comprising the step of:
   drilling a cortical area with a single drill up to a depth, said single drill having a diameter approximately equal to a widest diameter of the non-threaded dental implant and said depth corresponding to a total length of the non-threaded dental implant.
12. The method of item 11, wherein the single drill is made of tungsten carbide.
13. A kit for drilling bone for a dental implant (58) having a threaded portion and non-threaded portion, the kit comprising:
   a first drill (51) for drilling a cortical area (53) up to a first depth, said first drill (51) having a diameter (D1) approximately equal to a widest diameter of the dental implant (58) and said first depth corresponding to a length of the non-threaded portion of the dental implant (58); and
   a second drill (52) for drilling a borehole (54) into said cortical area (53) up to a second depth, said second drill (52) having a smaller diameter (D2) than the first drill (51) that is approximately equal to a difference between the widest diameter and a depth of threads of the dental implant (58), and said second depth corresponding to a length of the threaded portion (50) of the dental implant (58), such that the sum of the first depth and the second depth is approximately equal to the total length of the dental implant (58).
14. The kit of item 13, wherein the first drill (51) and/or the second drill (52) are made of tungsten carbide.
15. The kit of item 13 or 14, wherein the first drill (51) comprises a first stopper (70) and/or the second drill (52) comprises a second stopper (75).
16. The kit of item 15, further comprising a guiding tube (80) for use in combination with the stoppers (70, 75), to guide the drills (51, 52) during drilling.
17. Use of the kit of item 13 for drilling bone for the dental implant (58).
18. A kit for drilling bone for a non-threaded dental implant, the kit comprising: a single drill up for drilling a cortical area with to a depth, said single drill having a diameter approximately equal to a widest diameter of the non-threaded dental implant and said depth corresponding to a total length of the non-threaded dental implant.
19. The kit of item 18, wherein the single drill is made of tungsten carbide.
20. Use of the kit of item 18 or 19 for drilling bone for the non-threaded dental implant.

### BRIEF DESCRIPTION OF THE DRAWINGS

FIG. 1 shows a prior art drilling chart that illustrates the sequence and all the necessary drills for a ø 5.2 mm×10 mm implant.

FIG. 2 shows a schematic diagram that explains conventional ascending incremental drilling.

FIG. 3 shows a drill bit that has drilled into a cross-sectional view of cortical bone according to a step of an embodiment of the method of the present invention.

FIG. 4 shows a drill bit that has drilled into a cross-sectional view of medullary bone according to another step of the method of FIG. 3.

FIG. 5 shows a dental implant that has been implanted into a cross-sectional view of the borehole of FIG. 4.

FIG. 6 shows a dental implant, specifically dental implant elements, that can be used with an embodiment of the method of the present invention.

FIG. 7 shows a tungsten carbide (WC) drill bit, according to an embodiment of the present invention.

FIG. 8 shows a tungsten carbide (WC) drill bit according to an embodiment of the present invention about to be inserted into a cross-sectional view of cortical bone.

FIG. 9 shows the drill bit of FIG. 8, being inserted into a cross-sectional view of the cortical bone.

FIG. 10 shows a tungsten carbide (WC) drill bit according to another embodiment of the present invention being inserted into a cross-sectional view of medullary bone.

FIG. 11 shows the drill bit of FIG. 10, being inserted into a cross-sectional view of the medullary bone.

FIG. 12 shows the drill bit of FIG. 10, being removed from a cross-sectional view of the medullary bone.

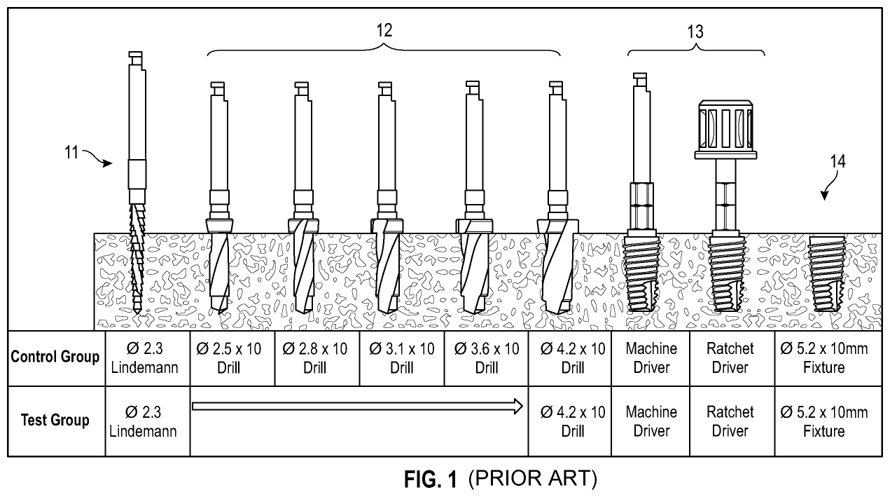

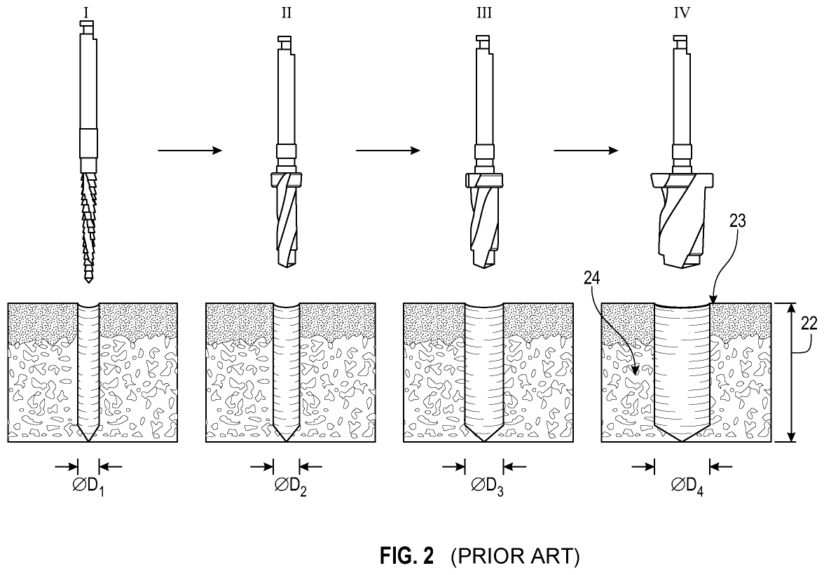

### DETAILED DESCRIPTION OF THE ILLUSTRATIVE EMBODIMENTS

### Method of Drilling Bone for Dental Implantation

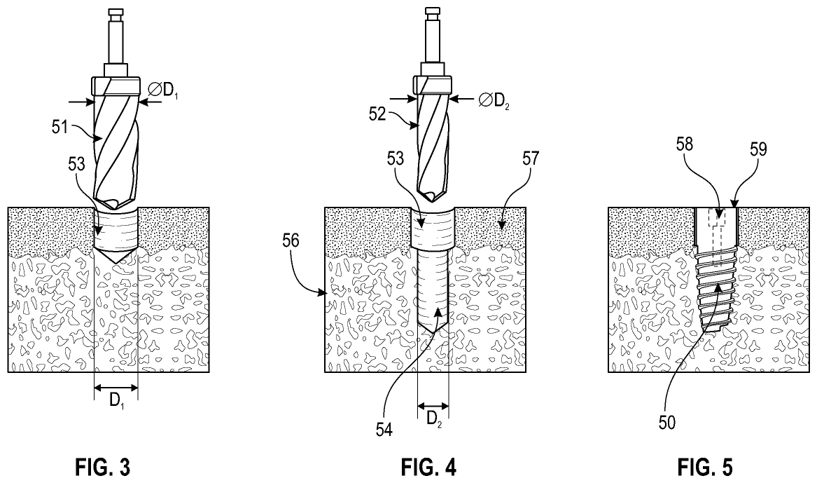

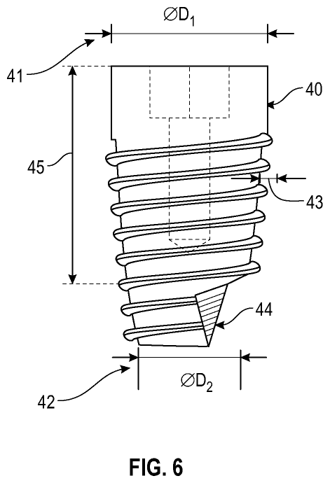

In a first aspect of the present invention, a method of drilling bone for a dental implant is provided. Referring first to FIGS. 3-5, as well as FIG. 6, this method of drilling bone for dental implantation will be described. As shown in FIGS. 3-5, said method, called the “Crown Down” method, comprises the steps of:
drilling a cortical area 53 with a first drill 51 up to a first depth (as shown in FIG. 3), said first drill 51 having a diameter D1 approximately equal to a widest diameter of the dental implant 58 and said first depth corresponding to a length of a non-threaded portion of the dental implant 58;
drilling a borehole 54 into said cortical area 53 with a second drill 52 up to a second depth (as shown in FIG. 4), said second drill 52 having a smaller diameter D2 than the first drill 51 that is approximately equal to a difference between the widest diameter and a depth of threads of the dental implant 58, and said second depth corresponding to a length of a threaded portion 50 of the dental implant 58, such that the sum of the first depth and the second depth is approximately equal to the total length of the dental implant 58 (as shown in FIG. 5).

As stated above, FIGS. 3-5 shows an embodiment of the “Crown Down” drilling method of the present invention. As mentioned, the first drill 51, comprising the final implant diameter, is used to drill the cortical area 53 of the cortex bone 57 (as shown in FIG. 3), followed by the smaller diameter (second) drill 52 being used to drill the rest of the depth (as shown in FIG. 4), thereby creating a borehole 54 for the implant 58 to self tap the medullar bone 56 using the threaded apical end 50 (as shown in FIG. 5). Once the implant 58 is inserted, the cortical bone and the cervical end 59 (i.e. widest portion) of the implant experience less (and are preferably free from) stress caused by pressing against each other, as opposed to with conventional drilling methods.

Table 1 below shows a drilling chart for examples of differently-sized BL Straumann implants for use with the method of the present invention. Table 1 also shows the drilling sequence and drilling depth that can be used with each implant.

TABLE 1
Crown Down drilling chart for dental implants

| | | Final drill |
|---|---|---|
| | | Final depth = |
| | | first depth + |
| | Pilot drill | second depth = |
| | First depth = | length of |
| Implant | 5 mm | implant |

| | | | | |
|---|---|---|---|---|
| 3.3 mm | 3.30 | mm | 3.00 | mm |
| 4.1 mm | 4.1 | mm | 3.75 | mm |
| 4.8 mm | 4.80 | mm | 4.3 | mm |

As can be seen in Table 1, for each implant diameter there are two drills. The first drill is used to drill the first 5 mm of depth, while the second one with a smaller diameter is used to drill the rest of the depth. The first drill has a diameter at least equal to the diameter of the implant. The skilled person would understand that other drills can be used than those shown in Table 1, and that the diameters of said drills will depend on the diameter and shape of the implant.

Moreover, the skilled person would understand that the first depth of 5 mm is merely a preferred embodiment, and that the first depth will also depend on the length of the non-threaded portion of the implant, as discussed in more detail below.

FIG. 6 shows the different areas of a dental implant that can be used with the method of the present invention. Factors that influence the drilling parameters include: 1) the length of the non threaded implant area 40 determines the depth of the first drilling; 2) the widest diameter of the dental implant is the greater diameter at the bone level and determines the diameter of the first drill; and 3) the second drill diameter is approximately equal to the smaller diameter 42 of the dental implant, which is the difference between the widest diameter 41 of the dental implant and the depth of the threads 43. The depth of the drilling with the second drill, when added to the depth of the first drilling, is equal to the length of the dental implant.

Implants comprising self drilling grooves 44 require shorter drilling, up to the level at which the grooves begin 45.

The first drill 51 and/or the second drill 52 are preferably made of materials that better exchange heat so as to cool down the drilling site, such as Tungsten Carbide. By using such a material, overheating is better prevented.

The first drill 51 and/or the second drill 52 preferably have higher mass to better absorb the heat resulting from the friction between the bone, the bone chips, and the active end of the drill. Such higher mass materials include Tungsten Carbide. By using such a material, overheating is better prevented.

In preferred embodiments, the first drill 51 and/or the second drill 52 operate with a speed of about 200 RPM (compared to conventional RMP of 800-1200 RPM using steel drills of the prior art). In preferred embodiments, the method of the present invention does not require irrigation (for example, the first drill 51 and the second drill 52 may have Tungsten Carbide drill bits). It is well known in the field that the temperature should not exceed 47 degrees Celsius. By using the present method with the drill made of Carbide, the operator can ensure that the temperature of 47 degrees Celsius is not exceeded.

In preferred embodiments, the first drill 51 and/or the second drill 52 can comprise a drill bit as defined in the subsequent section.

For clarity, Table 2 below provides a comparison between a preferred embodiment of the “Crown Down” method of the present invention and the prior art “incremental” technique.

TABLE 2
“Crown Down” Method vs Prior Art “Incremental” Technique

| Crown Down Method | Prior art “Incremental” Technique |
|---|---|
| Decrease in diameter | Increase in diameter |
| Progressive depth | One depth |
| 2 drills | 4 or more drills |
| 200 RPM | 1200-1500 RPM |
| Solid Carbide | High Speed Steel/Zirconium |
| No irrigation | Irrigation |

The skilled person would understand that the method of the present invention can work with conventional drills and can be used to create bore holes for a variety of dental implants.

The first drill, second drill, and/or the dental implant can be sold as part of a kit.

In preferred embodiments, the drills used in the method of the present invention are made of high thermo-conductivity material that have the capacity to absorb the heat resulting from the friction of the cutting edge of the tool with the bone.

As mentioned, by going from a bigger drill to a smaller one, the method of the present invention can facilitate the heat exchange between the bone and the drill.

In preferred embodiments, the drill bits used with the method comprise a larger surface area and greater mass, and an increased capacity to absorb heat, which can help the bones remain healthy and undamaged.

Histological analysis and in-situ micro-pillar compression tests revealed that high temperature drilling produces an 8-times larger necrotic depth (419±33.2 μm, n=185), when compared to low temperature drilling (49±3.9 μm, n=162), demonstrating that, in general, the higher the temperature, the greater the necrotic damage. Furthermore, in high temperature drilling, micro-pillar failure mode analysis revealed that the microstructure shifts from being ductile in the bulk, to brittle and mechanically weaker (up to −42% reduction in elastic modulus, −41% in ultimate compressive strength and −15% in yield strength) near the machined surface. It was found that this brittle layer can extend to at least 1500 μm away from the machined surface, which is more than 3 times the aforementioned necrotic depth. This brittle layer was found to be virtually inexistent in low temperature drilling, where micro-pillars in the necrotic layer retained both their pristine properties and their ductile failure mode.

During the drilling process of the bone, most of the heat generates at the drill cutting edge. That heat, if it persists, raises the temperature of the bone in situ, and can cause necrosis of the bone. In preferred embodiments, the drill bits used in the method of the present invention drive the heat away from the bone into the body of the drill bit, thereby protecting the bone from being damaged and reducing the temperature of the cutting site. Damaged bone from excessive heat leads to granulation tissue formation, and soft and hard tissue inflammation called Peri-Implantitis.

According to many publications, the prevalence of Peri-Implantitis over an average follow up of 2 years occurs in more than 30% of patients. Although Peri-Implantitis is an implant complication that can be attributed to multiple factors, one of the causes is damage to the bone by the heat during the drilling. Accordingly, one manner in which one can avoid that condition, especially when it occurs in the early stages after the implantation, is to prevent the bone from being overheated.

The primary stability of a dental implant is one of the main preconditions for osseointegration; in fact, micro-movement that exceeds the threshold of 100-150 microns can stimulate the growth of fibrous tissue in the bone to the implant interface, leading to the failure of the procedure.

In preferred embodiments, the method of the present invention increases primary stability by five times when compared to conventional methods.

In embodiments of the method of the present invention, the torque of the dental implant can be controlled by altering the diameter of the second drill (thereby altering the diameter of the borehole). For example, if the medullar area is particularly hard, the second drill can have a slightly larger diameter D2, which will lessen the torque when installing the dental implant. Conversely, if the medullar area is particularly soft, the second drill can have a slightly smaller diameter D2, meaning there is more torque when the dental implant is screwed in, as the resulting borehole will be slightly smaller.

In preferred embodiments, the method of the present invention allows the surgeon to control the torque from 0 up to 290 N/cm. “Over-drilling” (making the smaller diameter D2 slightly larger, as described above) with the second drill reduces the torque while “under-drilling” (making the smaller diameter D2 slightly smaller as described above) increases it.

In embodiments, the method of the present invention is simple and effective.

In embodiments, no matter the implant diameter, the method of the present invention needs no more than 2 drills. Furthermore, as mentioned, in embodiments, RPM can be around 200 and no irrigation is required.

Irrigation brings its own drawbacks. When drilling to create an osteotomy, autologous bone chips and osseous coagulum-a blend of blood, bone cells and growth factors—are created. Because of their osteogenic properties, when left in situ this material can promote new bone formation.

However, copious irrigation washes away bone chips and osseous coagulum. Once the bone chips are removed from the osteotomy, cells quickly begin to die, and so, even if retrieved, their osteogenic potential can quickly deteriorate. To maximize the healing potential of bone chips, the cell death should be minimized while maintaining bone chips in situ.

As can be seen in the following table, the method of the present invention can present several advantages when compared to methods of inserting dental implants using other drills. Specifically, Table 3 shows a comparison between a preferred embodiment of the method of the present invention and a method of inserting dental implants using N1 Osseoshaper.

TABLE 3
Comparison between preferred embodiment of method
of present invention and method of inserting
dental implants using N1 Osseoshaper

| | Embodiment of the Crown | |
|---|---|---|
| | Down method present | |
| Features | invention | N1 Osseoshaper |
| Recommended RPM | 200 | 50 |
| Diameters Ø mm | 2.5, 3.0, 3.3, 3.5, 3.75, 4.0, | 3.5, 4.0 |
| | 4.3, 4.5, 4.8, 5.0, 5.5, 6.0 | |
| Number of uses | More than 1000 times | Max. 20 times |
| Price USD | $189.00 | $90 |
| Material | WC | Stainless Steel |
| Thermal conductivity | 110 W/(m*K) | 20 W/(m*K) |
| Irrigation | Free | Free |
| Bone type | D1, D2, D3, D4 | Unknown |

In addition to the advantages discussed above, in embodiments, the method of the present invention can present one or more of the following advantages:
Superior primary implant stability
2 drills instead of 7 conventional drills
Increased reusage, compared with 40 drilling cycles.
No need for irrigation
Prevents tissue inflammation and immediate and late implant failure.
Compatible with all implant systems
Saves up to 80% on working time
Easy to clean and sterilize
Predictable success.
Implants placed with “Crown Down” drills can be loaded 5 times faster (4 weeks instead of 5 months)

### Drill Bit for Dental Implantation

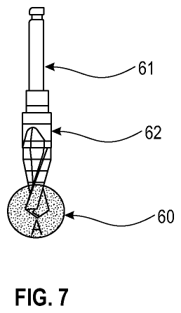

In a second aspect of the present invention, a drill bit is provided. Referring first to FIG. 7, the drill bit, generally referred to using the reference numeral 61, will be described.

Referring to FIG. 7, there is shown a tungsten carbide (WC) drill bit 61 according to an embodiment of the present invention. As persons skilled in the art will understand, what generates most of the heat is the tip 60 of the drill bit 61 compared to the body 62. This energy may be represented by the following formula:

Q=k*A*dT
where:
Q=Energy flow [W]
k=Overall heat transfer coefficient [W/m²×° C.]
A=Heat transfer area [m²]
dT=Temperature difference [C]

In general, the thermal conductivity of WC Tungsten Carbide is 110 W/m²k.

The thermal conductivity of HSS High Speed Steel is 18-23 W/m²k.

The thermal conductivity of Zr Zirconium is 11 W/m²k.

In preferred embodiments, at least one drill bit of the present invention is used in the method defined in the previous section as part of either the first drill 51, the second drill 52, or both.

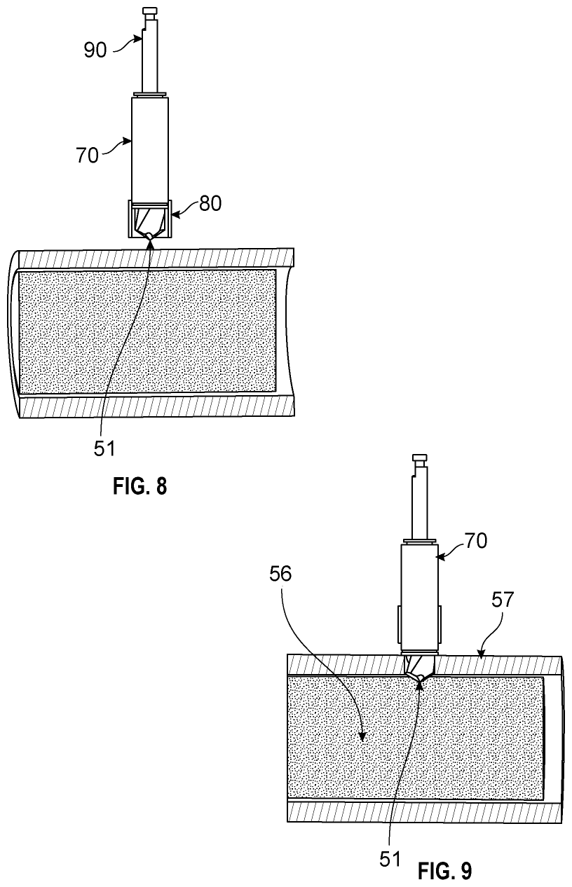

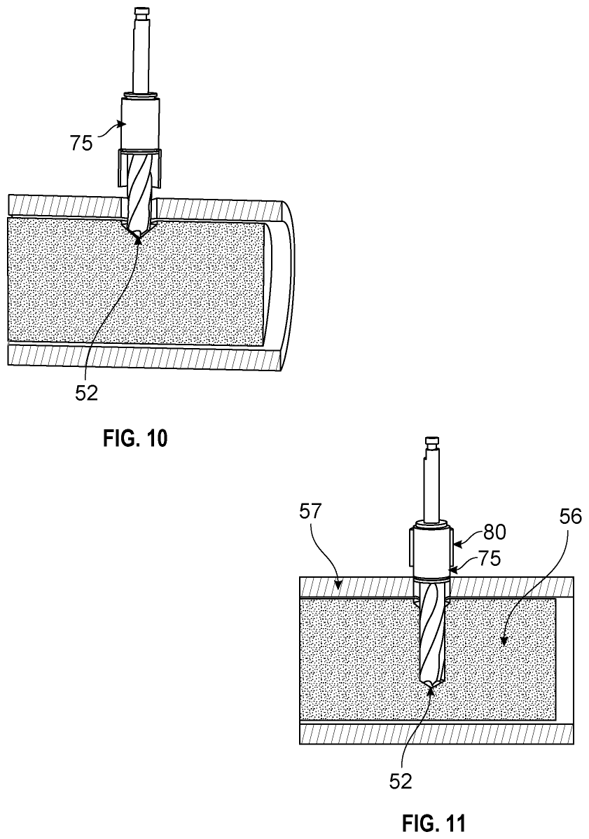

FIGS. 8-12 show drill bits 61 according to a preferred embodiment of the present invention being used in an embodiment of the method of the present invention (as defined in the previous section). FIGS. 8-9 show the first drill 51 drilling into a cross-sectional view of the cortical bone 57 up to a first depth. FIGS. 10-11 show the second drill 52 drilling a borehole 54 into a cross-sectional view of the medullary bone 56 up to the second depth, said second drill 52 having a smaller diameter than the first drill 51. As can be seen in FIGS. 8-11, the first drill 51 can comprise a first stopper 70, and the second drill 52 can comprise a second stopper 75, which can be used to ensure accurate depth control, which can help ensure that the desired depth is reached consistently.

Moreover, a guiding tube 80, a cross-section of which can be seen in FIGS. 8-12, can be used with the first drill 51 and second drill 52. The guiding tube 80, when used in combination with stoppers 70 75, guides the drills 51 52 during drilling, and each stopper 70 75 gets slidably pushed along the guiding tube 80 as each drill bores further into the cortical bone 57 or the medullary bone 56. Specifically, the external surface of each stopper 70 75 is configured to guide the drills 51 52 with the inner surface of the guiding tube 80. Conventionally, guiding tubes of different sizes are needed to guide drill bits of different sizes, meaning a differently-sized guiding tube needed to be used for each drill bit of different diameter. However, in preferred embodiments of the present invention, by having the first stopper 70 have the same diameter as the second stopper 75, as shown in FIGS. 8-12, a single guiding tube 80 can be used for both the first drill 51 and second drill 52. The length and position of the guiding tube 80, as well as the length of each stoper 70 75, can be selected such that each stopper 70 75 starts to be guided by the guiding tube 80 as drilling commences (as shown in FIGS. 8 and 10).

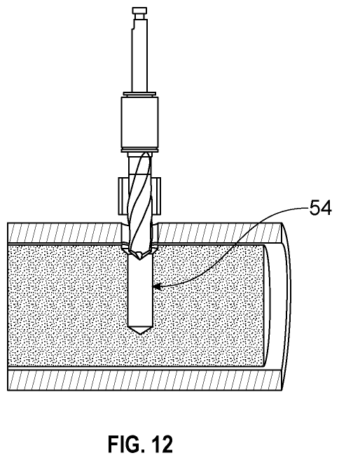

As can be seen in FIG. 8, the first drill 51 also comprises a shank 90, which can be used to connect the drill to a rotary tool. In FIG. 12, the second drill 52 is being removed from the borehole 54, leaving the osteotomy borehole 54 into which a dental implant can be inserted.

In embodiments, the drill bit of the present invention protects the bone from being overheated, thereby helping ensure predictable implant results.

In preferred embodiments, the drill bits of the present invention are built from a very hard and resistant metal for an increased number of uses. As an example, using the drill bit shown in FIG. 7, after 1000 drilling cycles (using bovine rib drilling cycle tests, where the drill bits were tested repeatedly on bovine ribs), there were absolutely no signs of wear observed.

The scope of the claims should not be limited by the preferred embodiments set forth in the examples, but should be given the broadest interpretation consistent with the description as a whole.

### Definitions

The use of the terms “a” and “an” and “the” and similar referents in the context of describing the invention (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise claimed.

No language in the specification should be construed as indicating any non-claimed element as essential to the practice of the invention.

Herein, the term “about” has its ordinary meaning. In embodiments, it may mean plus or minus 10% or plus or minus 5% of the numerical value qualified.

Unless otherwise defined, all technical and scientific terms used herein have the same meaning as commonly understood by one of ordinary skill in the art to which this invention belongs.

1-20. (canceled)

21. A method of drilling bone for a dental implant having a threaded portion and non-threaded portion, the method comprising steps of:
drilling a cortical area with a first drill up to a first depth, said first drill having a diameter approximately equal to a widest diameter of the dental implant and said first depth corresponding to a length of the non-threaded portion of the dental implant;
drilling a borehole into said cortical area with a second drill up to a second depth, said second drill having a smaller diameter than the first drill that is approximately equal to a difference between the widest diameter and a depth of threads of the dental implant, and said second depth corresponding to a length of the threaded portion of the dental implant, such that the sum of the first depth and the second depth is approximately equal to the total length of the dental implant.

22. The method of claim 21, wherein, once the dental implant is inserted, the cortical bone and the cervical end of the implant experience less stress caused by pressing against each other.

23. The method of claim 21, wherein the first drill and/or the second drill have larger surface area to better absorb the heat resulting from the friction between the bone, the bone chips, and the active end of the drill.

24. The method of claim 21, wherein the first drill and/or the second drill are made of tungsten carbide.

25. The method of claim 21, wherein the first drill and/or the second drill operate with a speed of about 200 RPM.

26. The method of claim 21, wherein the method is free of irrigation.

27. The method of claim 21, wherein the first drill comprises a first stopper and/or the second drill comprises a second stopper.

28. The method of claim 27, wherein a guiding tube is used in combination with the stoppers, to guide the drills during drilling.

29. A kit for drilling bone for a dental implant having a threaded portion and non-threaded portion, the kit comprising:
a first drill for drilling in a cortical bone of a cortical area up to a first depth, said first drill having a diameter equal to or wider than a widest diameter of the dental implant and said first depth corresponding to a length of the non-threaded portion of the dental implant; and
a second drill for drilling a borehole in a medullary bone into said cortical area up to a second depth after drilling the cortical bone up to the first depth with the first drill, said second drill having a smaller diameter than the first drill that is approximately equal to a difference between the widest diameter and a depth of threads of the dental implant, and said second depth corresponding to a length of the threaded portion of the dental implant, such that the sum of the first depth and the second depth is approximately equal to the total length of the dental implant, such that the sum of the first depth and the second depth is approximately equal to the total length of the dental implant, such that the sum of the first depth and the second depth is approximately equal to the total length of the dental implant.

30. The kit of claim 29, wherein the first drill and/or the second drill are made of tungsten carbide.

31. The kit of claim 29, wherein the first drill comprises a first stopper and/or the second drill comprises a second stopper, the kit further comprising a guiding tube, wherein each stopper is configured to get slidably pushed along the guiding tube during use of the drills, and wherein an external surface of each stopper is configured to guide the drills along an inner surface of the guiding tube during use thereof.

32. A method of drilling bone for a non-threaded dental implant, the method comprising drilling a cortical area with a single drill up to a depth, said single drill having a diameter approximately equal to a widest diameter of the non-threaded dental implant and said depth corresponding to a total length of the non-threaded dental implant.

33. The method of claim 32, wherein the single drill is made of tungsten carbide.
